(19) United States
(12) Patent Application Publication
(10) Pub. No.: US 2007/0010824 A1
(43) Pub. Date: Jan. 11, 2007

(54) Products, systems and methods for delivering material to bone and other internal body parts

(76) Inventors: Hugues Malandain, Mountain View, CA (US); James Rappaport, Reno, NV (US)

Correspondence Address: KILPATRICK STOCKTON LLP - 55461, 1001 WEST FOURTH STREET, WINSTON-SALEM, NC, 27101, US

(21) Appl. No.: 11/410,182

(22) Filed: Apr. 24, 2006

Related U.S. Application Data
(60) Provisional application No. 60/698,291, filed on Jul. 11, 2005.

(51) Int. Cl. A61F 2/00

(52) U.S. Cl. 606/92

(57) ABSTRACT

Disclosed are products, systems, and methods for delivering a material to an internal body part such as bone. In one variation, the product comprises a segmented plunger having a plurality of segments, where the plurality of cylindrical segments are sized for urging a material through an access member to thereby deliver the material to predetermined location in a subject's body. The segmented plunger may be used with a catheter for delivering a bone filler or a bone cement to a bone requiring repair.

### STATEMENT OF RELATED APPLICATIONS

The present application claims priority under 35 USC 119(e) to U.S. provisional application No. 60/698,291, filed Jul. 11, 2005, entitled “Devices, Systems And Methods For Emplacing Material In Bone And Other Internal Body Structures.” The disclosure of provisional patent application 60/698,291 is hereby incorporated by reference in its entirety herein.

### FIELD OF INVENTION

The present invention relates to products, systems, and methods for delivering a material to an internal body part such as bone.

### BACKGROUND

A variety of conditions may warrant repair and/or replacement of an internal body part, such as bone. For example, to repair a bone fracture, an adhesive agent may be applied to adhere sections of the separated bone together. Also, a bone filler material may applied to a bone to replace degenerated tissue and/or to provide a supportive matrix to support or reinforce the bone.

In many instances, it can be preferred to minimize the invasiveness of a procedure used for treatment and repair of an internal body part, such as bone. Minimally invasive spine fracture surgeries may be performed by accessing the fractured bone using a cannula-based technology. For example, a cannula-based bone filler device may comprise an outer cannula and an inner rod-like tamping instrument. The cannula may be loaded with an aliquot of a bone repair material using an injection nozzle and syringe, and the repair material urged to the site requiring repair using the tamping instrument.

To optimize emplacement of the correct amount of a bone repair material into the bone, the repair material may utilize a radiopaque tracer to allow for visualization of the repair material as it is being emplaced. For example, the patient may be positioned in an X-ray apparatus known as a C-arm, with the X-ray transmitter (one arm of the “C”) above the patient, and the receiver (the other arm of the “C”) underneath the patient. Optimal positioning of the C-arm (or other radio-imaging device) may require that the imaging device be positioned very close to the site that is being treated. For example, optimal positioning of the C-arm may require that the arms of the C-arm device be only a few inches from the patient's torso.

Generally, the cannula used with a bone filler device must be long enough to reach from the bone that is being treated to the patient's exterior, so as to be accessible to the physician. Also, to completely expel all of the material loaded in the cannula, the rod-like tamping instrument must be as long as the cannula. Where the tamping instrument is substantially straight, this can require that there is a clearance from the patient's torso that is at least as large as the length of the tamping instrument, if not larger. However, if radio-imaging is being used to track emplacement of the repair material in the bone, there may only be limited access to the patient due to close positioning of the C-arm or other monitoring device.

Thus, there is a need to provide products that can be used to deliver therapeutic materials to the interior of a patient's body where access to the patient may be restricted. For example, there is a need to provide a bone filler device that can access a patient where the imaging device is positioned close to the patient's torso.

### SUMMARY OF THE INVENTION

Embodiments of the present invention comprise products, systems and methods for delivering material to a predetermined location in a subject, such as an internal body part or region. For example, in one embodiment, the present invention may comprise a product comprising a plunger having a plurality of segments sized for urging a material through an access member to a predetermined location within a subject.

Other embodiments and further details on various aspects of the present invention are set forth in the following description, figures, and claims. It is to be understood that the invention is not limited in its application to the details set forth in the following description, figures, and claims, but is capable of other embodiments and of being practiced or carried out in various ways.

### BRIEF DESCRIPTION OF THE FIGURES

FIG. 1 illustrates a prior art plunger and outer cylinder for a bone filler device.

FIG. 2, panels (A) and (B), illustrate a prior art plunger and outer cylinder being used to emplace a bone filler material in a bone.

FIG. 3 illustrates a segmented plunger for a bone filler device in accordance with an embodiment of the present invention where the plunger is in a straight conformation.

FIG. 4 illustrates a segmented plunger for a bone filler device where the plunger is in a bent conformation, where panel (A) shows a first conformation, and panel (B) shows a second conformation in accordance with alternate embodiments of the present invention.

FIG. 5 illustrates a segmented plunger for a bone filler device being pushed through a tube containing a bone filler material, where panels (A) and (B) illustrate progression of the plunger through a tube in accordance with alternate embodiments of the present invention.

FIG. 6 illustrates a tool for pushing the segments of a segmented plunger through a tube where panel (A) shows the distal surface of the tool, and panel (B) shows the proximal surface of the tool in accordance with an embodiment of the present invention.

FIG. 7 illustrates use of a tool for urging a segmented plunger through a tube comprising a material for delivery to a body part in accordance with an embodiment of the present invention.

FIG. 8 illustrates a segmented plunger for a bone filler device in accordance with an alternate embodiment of the present invention where the segments of the plunger are not connected to each other.

FIG. 9 illustrates a view of a segmented bone filler device having segments that are not connected to each other being pushed through a tube in accordance with an embodiment of the present invention.

FIG. 10, panels (A) and (B), illustrate two systems in accordance with alternate embodiments of the present invention using either a plunger having connected segments (panels A1-A4) or a plunger having segments that are not connected (panels B1-B4).

FIG. 11 illustrates a view of a segmented plunger having segments that are not connected being pushed through a tube having an exit port on the side of the tube in accordance with an alternate embodiment of the present invention.

FIG. 12 illustrates a kit comprising a segmented plunger in accordance with an embodiment of the present invention.

FIG. 13 illustrates a method for using a segmented plunger as part of a bone filler device in accordance with an embodiment of the present invention.

### DETAILED DESCRIPTION

As used herein, a predetermined location in a subject may comprise any region inside a body or body part. As used herein, an internal body part may comprise bone, cartilage, tissue, or an internal organ, or parts thereof. A body part may comprise a bone or bones (e.g., a vertebral body or spinal disc), a cartilage, a tendon, muscle, a vein, an artery, or an organ (e.g., intestines, stomach, liver or lung), or part thereof, that may need, for example, to be accessed and repaired. Or, the predetermined location may comprise an internal body region, such as the vasculature, abdomen, or other body regions. Furthermore, in this specification and the appended claims, the singular forms “a,” “an” and “the” include plural referents unless the context clearly dictates otherwise. Thus, for example, the term “a lumen” is intended to mean a single lumen or a combination of lumens, “a fluid” is intended to mean one or more fluids, or a mixture thereof. In addition, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body. Thus, for example, the cannula end inserted inside the patient's body would be the distal end of the cannula, while the cannula end outside the patient's body would be the proximal end of the cannula.

Also, as used herein, an access member comprises a device for accessing a predetermined location in a subject. Thus, the access member may provide a path to access a region or a body part that is located within the subject's body. The access member may be any type of device that can extend from the location of interest (e.g., a bone or an organ) to be accessible to a user of the access member. For example, the access member may be designed to extend from an internal body part in a subject to outside of the subject's body. The access member may be an elongated hollow member such as a hollow cylinder or tube.

Also, as used herein, a material for emplacement within, or delivery to, a predetermined location in a subject may comprise any material that is biologically compatible with the predetermined location of interest. For example, in alternate embodiments, the material may comprise a bone filler material, an adhesive, a therapeutic drug, a tissue graft, a population of cells, a biological matrix, or any other physiological material for delivery to a location in a body. As used herein, a bone filler material comprises any material that may be used for the treatment of bone. A variety of materials have been described for use as bone filler materials (see e.g., U.S. Pat. Nos. 4,904,257, 6,203,574, 6,579,532, 6,740,093, and Patent Application No. 2005/0136038 for descriptions of bone filler materials, and each of which is incorporated herein by reference in its entirety for all purposes). In one embodiment, the bone filler or treatment material may comprise an adhesive.

Embodiments of the present invention provide products, systems, and methods for delivering a material to a predetermined location in a subject, such as an internal body part or region. The present invention may provide access to internal body regions and/or internal body parts where there may be limited clearance to perform the delivery procedure. For example, many procedures for delivering a material to an internal body part require positioning of an X-ray apparatus close to the patient's torso for visual monitoring of the delivery process. Particular embodiments of the present invention may comprise products, systems, and methods to emplace a bone filler or other treatment material for the treatment and repair of bone.

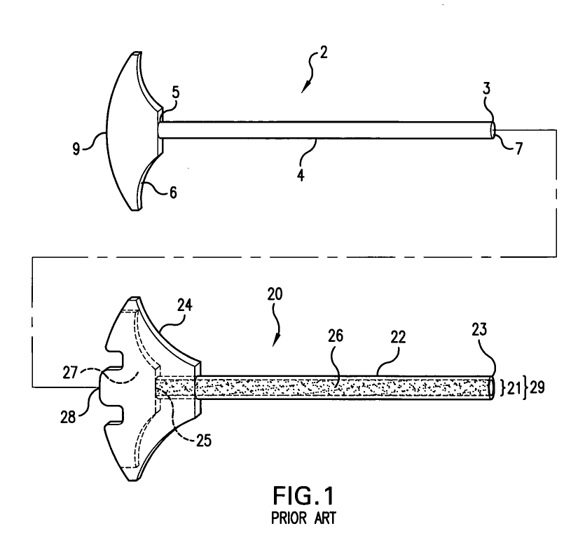

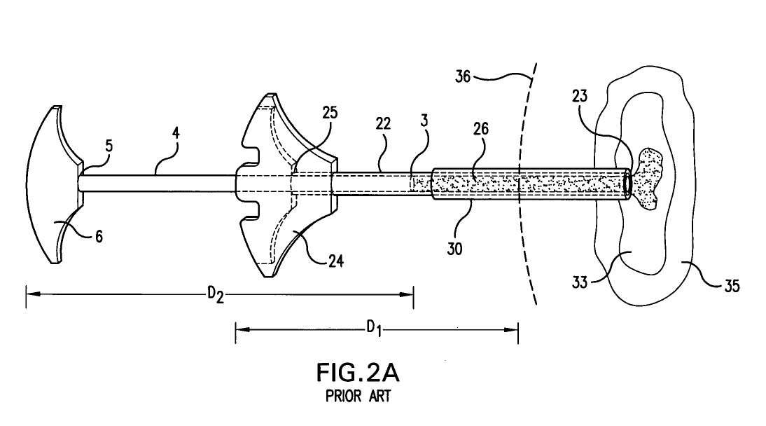

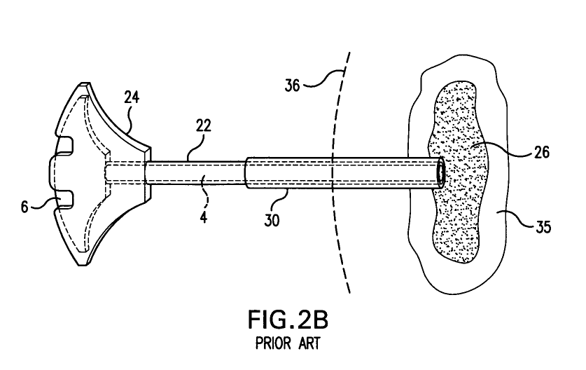

To better understand the present invention, a bone filler device of the prior art for delivery of bone filler material to a bone is shown in FIGS. 1 and 2. FIG. 1 shows the individual parts of the bone filler device, and FIG. 2 shows the device at two different positions during a procedure for emplacing bone filler material into a bone. Generally, the bone filler device comprises a first outer part 20, comprising a hollow cylinder or tube 22 and handle 24, and a second inner part 2, comprising a closed cylinder 4 and handle 6. The outer cylinder 22 comprises a distal end 23 (i.e., the end farthest from the user), and a proximal end 25 (i.e., the end closest to the user). Also, the inner cylinder 4 comprises a distal end 3 and a proximal end 5.

As illustrated in FIGS. 1 and 2, the two concentric cylinders 4, 22, may be used to emplace a paste-like bone filler material 26 into the bone. The open interior 21 of the outer cylinder 22 of the bone filler device is designed to provide access to the bone being repaired by providing a cannula-type access into which the inner cylinder 4 fits. The inner cylinder 4 generally has the distal end 3 closed off to form a substantially flat surface 7. To emplace the bone filler material, the outer cylinder 22 may be filled with the required bone filler material 26. Generally the outer cylinder will be loaded with enough bone filler material so that the cylinder comprises material from the distal end 23 to the proximal end 25. In some cases, however, the outer cylinder is only partially filled with the bone filler material. The filled outer cylinder may then be inserted into the bone, usually through a second cylindrical cannula 30 having a slightly larger inner diameter than the diameter 29 of the outer cylinder 22 of the bone filler device (FIG. 2).

For example, as illustrated in FIGS. 2A and 2B, where a catheter and balloon have been used to compact bone and to create a internal void space 33 in a bone 35 requiring repair, the outer cylinder 22 of the bone filler device may be emplaced in a cannula tube 30 that is positioned in the bone. The inner cylinder 4 is then inserted into the proximal end 25 of the outer cylinder 22 (i.e., the end of the device that is closest to the user). As illustrated in FIGS. 1, 2A, and 2B, the inner part 2 of the bone filler device may comprise a handle 6 that can fit within the handle 24 of the outer part 20 of the bone filler device. Thus, to emplace the filler material 26 in a bone positioned at the distal end 23 of the bone filler device, the distal end 3 of the inner cylinder 4 is inserted into the proximal end 25 of the outer cylinder 22 (FIG. 2A), and is then pushed through the outer cylinder to urge the bone filler material 26 through the outer cylinder 22 and into the bone cavity 33 in the bone 35 to be treated (FIG. 2B). The inner part 2 may be pushed through the outer part 20 until the distal end 3 of the inner part 2 is substantially flush with the distal end 23 (i.e., the end that is farthest from the user) of the outer part 20 (FIG. 2B). To allow for complete removal of the bone filler material 26 from the outer cylinder 22, there may be a chamber 27 (see e.g., FIG. 1) in the handle 24 of the outer part shaped to allow for the inner handle 6 to sit within the chamber when the inner cylinder 4 has passed entirely through the outer cylinder 22 (see e.g., FIG. 2B). For example, the bone filler device may be designed such that the proximal end 9 of the inner handle 6 may be substantially flush with the proximal end 28 of outer handle 24 when the two distal ends 3, 23 of the inner and outer cylinders are aligned (see e.g., FIG. 1 and FIG. 2B).

Because the bone filler device is comprised of two straight cylindrical pieces (i.e., inner cylinder 4 and outer cylinder 22), the user of the device may require sufficient clearance to allow insertion of the inner cylinder 4 into the outer cylinder 22. It may be seen from FIGS. 2A and 2B that the bone filler device may require a clearance that is equal to the sum of the distance D₁ that the outer part 20 extends from the surface 36 of the patient being treated and the length D₂ of the inner part 2. As described above, to ensure that the bone filler material is correctly emplaced, a radio-opaque fluorophore may be added to the filler material and the process of filling the bone monitored by transmission of X-rays using a C-arm apparatus. Optimal positioning of the C-arm (or other radio-imaging device) may require that the X-ray device be positioned as close to the patient as possible. For example, optimal positioning of the C-arm may require that the upper arm of the C-arm device be only inches from the patient's torso. Close positioning of the C arm, however, can restrict the physician's ability to insert a long inner part 2 of the bone filler device into the outer part 20 of the bone filler device.

Embodiments of the present invention may provide products, systems, kits and methods to access an inner body part where there is restricted clearance for the physician to insert an invasive device. In one embodiment, the product comprises a plunger having a plurality of segments sized for urging a material through an access member to a predetermined location in a subject. In an embodiment, the access member comprises a path for delivering the material to the predetermined location.

In one embodiment, the subject is an animal. For example, the subject may comprise a mammal. In one embodiment, the subject may be a human. The user of the product requiring access to the subject may be a physician, veterinarian, or other type of health care professional. In another embodiment, however, a user of the product may be accessing a particular location in his or her own body, as for example, for periodic delivery of a therapeutic material.

The predetermined location may, in certain embodiments, comprise a body part within a living body. In an embodiment, the predetermined location may comprise a bone. In one embodiment, the predetermined location may comprise a bone interior. For example, the predetermined location may comprise a portion of a spine. Thus, in one embodiment, the plunger may be sized for urging a material through an access member to thereby deliver the material to a bone interior, such as a vertebral body or a spinal disc.

For example, due to various traumatic or pathologic conditions, such as osteoporosis, a vertebral body can experience a vertebral compression fracture (VCF). In such conditions, at least a part of the vertebral bone can be compacted, causing a decrease in height of the vertebra. In many cases, vertebral height is lost in the anterior region of the vertebral body. Thus, the products, methods, kits and systems of the present invention may by used to repair a vertebral body lost due to a fracture, or when other degeneration occurs. The present invention is not limited in application to vertebrae, and may be used to repair other parts of a living or non-living organism. For example, in embodiments, the products, methods, kits and systems of the present invention can be deployed in other bone types and within or adjacent other tissue types, such as in a vertebral disc, an arm bone, a leg bone, a knee joint, and the like.

As described herein, the access member may provide a path to access a region or a body part that is located within a subject's body. The access member may be any type of device that can extend from the location of interest (e.g., a bone or an organ) to be accessible to a user of the access member. For example, the access member may be designed to extend from an internal body part in a subject to outside of the subject's body. The access member may comprise an elongated hollow member such as a hollow cylinder or a tube. Thus, in one embodiment, the tube may be designed to provide an access from outside of a living body to the internal body part. In an embodiment, the segments of the plunger and the access member are substantially cylindrical in shape. For example, the access member may comprise a cannula, such as a cannula used to deliver a material to bone or another type of body part. One of ordinary skill in the art having the benefit of this disclosure would appreciate that the segments of the plunger and the access member can be configured with other shapes and/or dimensions such as oval, hexagonal, octagonal, and the like.

In an embodiment, the access member may be configured to provide percutaneous surgical access to the predetermined location. As used herein, a percutaneous surgical access denotes passage through substantially unbroken skin, as for example, by needle puncture, a cannula or a catheter. In alternate embodiments, the percutaneous surgical access may comprises an incision ranging from about 0.1 to 4.0 centimeters (cm) in diameter, or from about 0.2 to 2.0 cm in diameter, or from about 0.25 to 1 cm in diameter. Thus, in alternate embodiments, the percutaneous surgical access may comprise an incision that is less than 4 cm in diameter, or less than 2 cm in diameter, or less than 1 cm in diameter. In one example embodiment, the percutaneous surgical access may comprise an incision of about 1 cm in diameter.

For example, in a typical percutaneous surgical repair of a spine, a cannula may establish a percutaneous path along its elongated axis to a vertebral body of one of the several vertebrae. The vertebral body extends on the anterior (i.e., front or chest) side of the vertebrae. The vertebral body comprises an exterior formed from compact cortical bone. Cortical bone is bone consisting of, or relating to, the cortex or outer layer of a bony structure. The cortical bone may enclose an interior volume of reticulated cancellous or spongy, bone (also called medullary bone or trabecular bone). Cancellous bone is bone having a porous structure comprising many small cavities or cells. The vertebral body is in the shape of an oval disc, and access to the interior volume of the vertebral body can be achieved, for example, by drilling an access portal through a rear side of the vertebral body (a postero-lateral approach). The portal for the postero-lateral approach may enter at a posterior side of the vertebral body and extend anteriorly into the vertebral body. Alternatively, access into the interior volume of a vertebral body can be accomplished by drilling an access portal through one or both pedicles of the vertebra. This is known as a transpedicular approach.

In an embodiment, each of the plunger segments comprises a diameter less than the internal diameter of the access member and a length less than the length of the access member. In this way, the plurality of segments may be inserted into the access member and function to push a material that has been loaded in the access member from one end of the access member to the other end of the access member. Also, in an embodiment, the plurality of the segments together comprise a length that is less than, or substantially the same as, the length of the access member. For example, a plunger comprising three segments may have an overall length that is less than, or substantially the same as, the length of a tubular access member comprising a material for delivery to a body part. In this way, the plunger will not extend out the distal end of the tube after the plunger has been used to urge the material in the tube out the distal end of the tube for delivery to a body part.

The plurality of segments may be connected as one unit, or may be separate parts. Thus, in an embodiment, at least two of the segments are attached to each other by a connector. In an embodiment, the connector may be fixedly (i.e., such that the connector is not removable during normal use) attached to the segments. Alternatively, the connector may be removably attached to the segments. The connector may, in certain embodiments, comprise a material that is bendable. This may allow the plunger to assume various conformations as the segments are urged through the access member. Also, in an embodiment, the connector may comprise a material having shape memory. By having shape memory, a connector having a first conformation may be urged into a second conformation, and then regain its original (first) conformation as the plunger is urged through the tube. Additionally or alternatively, it may be important for the connector to provide structural strength and to maintain an overall linear conformation as the plunger urges the material for delivery to the body part through the access member. Thus, in an embodiment, the connector may comprise a substantially rigid material. In an embodiment, the connector is substantially straight.

In certain embodiments, the product of the present invention may comprise a plurality of segments sized for urging a material through an access member for delivery of a material to a predetermined location in a subject, where the segments are not connected to each other. For example, in one embodiment, the plurality of segments may be configured such that an end of a first segment can be positioned to be in direct communication with an end of an adjacent segment.

In an embodiment, the segmented plunger of the present invention may provide a means to deliver a material to a predetermined location in a subject, such as an internal body part, where the working environment comprises limited access. Thus, embodiments of the products of the present invention may further comprise at least one tool for urging at least one of the segments through the access member. In an embodiment, the segment being urged through the access member travels from the proximal end of the access member (i.e., the end closest to the user) towards the distal end of the access member (i.e., the end farthest from the user). The tool of the present invention may, in certain embodiments, comprise a size and shape to accommodate limited access to the subject being treated.

The tool may be used to urge the segments through the access member either individually, or as a linear unit of segments. In an embodiment, the tool may be configured to be in communication with an end of at least one of the plurality of segments. For example, the tool may be shaped to engage the proximal end of at least one of the plurality of segments. In alternate embodiments, the tool may either be fixedly attached to the segment, or the tool may be removably attached to the segment.

The tool may comprise various shapes depending upon the specific application for which the product of the present invention is being used. Also, in certain embodiments, a variety of different tools for urging at least one of the segments through the tube may be used. In one example embodiment, the tool may comprise a substantially flat surface configured to engage a surface of one of the segments. Also, the tool may be differently configured depending upon how it is used to urge segments through the access member. For example, in an embodiment, the tool may configured to engage the distal end of a segment that extends from the proximal end of the access member to push the segment towards the distal end of the access member. Alternatively, the tool may comprise a configuration designed to pull segments that are outside of an access member into the access member. For example, in an embodiment, the tool may comprise a means to wrap around a segment, so as to allow the tool to grab the segment and urge the segment in a particular direction. Or, the tool may comprise a means to attach to a fastener present on the end of a segment, thereby allowing the tool to be attached to the segment and urge the segment a particular direction.

In one embodiment, the tool may comprise a handle. Alternatively or additionally, the tool may be configured to be substantially planar in shape. In an alternate embodiments, the tool may comprise a substantially planar shape that is rectangular, polygonal (e.g., hexagonal, octagonal), circular, oval, or the like. For example, in one embodiment, the tool may comprise a disk-like shape.

In an embodiment, the tool for urging segments through an access member may be configured to have an aperture. The aperture may comprise a means for the tool to be positioned around a connector connecting a first segment to a second segment. For example, in one embodiment, the tool may comprise a disk-shaped device having an aperture that allows for the disk to wrap around a connector connecting a first segment to a second segment, such that a substantial portion of at least one surface of the disk engages a surface of the first segment.

The aperture portion of the tool may comprise a variety of shapes. In an embodiment, a portion of the aperture comprises a substantially circular shape having a larger circumference on the proximal surface of the tool than on the distal surface of the tool. For example, for a disk-shaped tool, a portion of the aperture may comprise a funnel-like shape having a wide opening to allow for bending of the connector that extends from the access member, but having a straight portion to align the distal end of the connector with the access member.

The tool may be shaped to allow for the user to easily manipulate the tool. Thus, the tool may comprise a handle. Additionally or alternatively, the tool may comprise a surface shaped to engage at least a portion of a user's hand. For example, the tool may comprise indented portions shaped to facilitate grasping the tool with one's hand. In an embodiment, the indented portions may comprise ribbing or other molding to enhance gripping of the tool by a user.

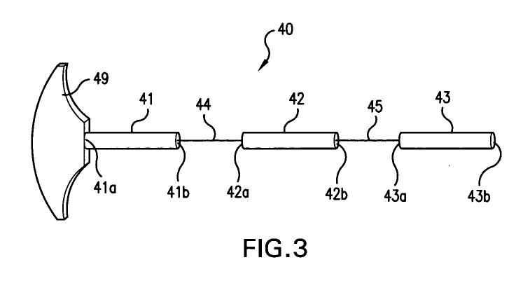

The present invention may be better understood by reference to the following non-limiting figures and description. FIG. 3 provides an illustrative representation of an embodiment of a product of the present invention. In an embodiment, the product may comprise a device that functions as a plunger for a bone filler device. As shown in FIG. 3, in an embodiment, a segmented plunger 40 of the present invention may comprise individual segments 41, 42, 43 that have a dimension so as to be inserted into an access member, such as a hollow tube. In an embodiment, the access member and the segments may be cylindrical in shape. Also, in an embodiment, the access member may comprise a cannula of a bone filler device (e.g., 20 of FIG. 1). In an embodiment, the plurality of the segments together have a length that is equal to, or less than, the length of the access member. For example, in alternate embodiments, two, three, four, or five or more, interconnected segments may comprise a segmented plunger having an overall length equal to, or less than the length of the access member. Or, as discussed below, the segmented plunger may comprise a plurality of segments that are not connected, but that together have an overall length equal to, or less than, the length of the access member. Thus in an embodiment, the segments may comprise interconnected segments that form a plunger-like apparatus that may be used to urge a material from a proximal end of a tube that is positioned outside of a patient's body, to the distal end of the tube that is positioned at the site of, or within, a location of interest in the body. In an embodiment, the product of the present invention may be used to deliver a bone repair material to a bone requiring repair. Or, the product may be employed for other types of cannula systems where access to the patient may be restricted.

In certain embodiments, the segments each comprise two closed ends 41a, 41b, 42a, 42b, 43a, 43b, so as to be closed cylinders. The segments may be solid, or they may be at least partially hollow. In an embodiment, the individual segments are constructed so as to be sufficiently rigid such that they do not bend when inserted into an access member, such as a tube, used to contain the material being delivered to a location of interest in a subject's body. Also, the segments may comprise a material that is compatible with the other parts of the system. Thus, in certain embodiments, the segments may be made of metal. For example, metals such as aluminum, stainless steel, spring steel, a nickel titanium alloy, or other alloys may be used. Or, the segments may be made of plastic. For example, a resilient plastic such as polypropylene, polyethylene, polyethylenetetraphthalate (PET), or nylon may be used. One of ordinary skill in the art having the benefit of this disclosure would appreciate that other materials, including those that are well-known to one in the art, may be applied to configure the segments described herein.

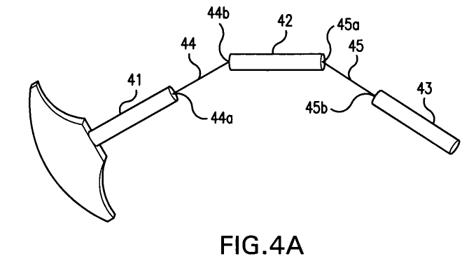

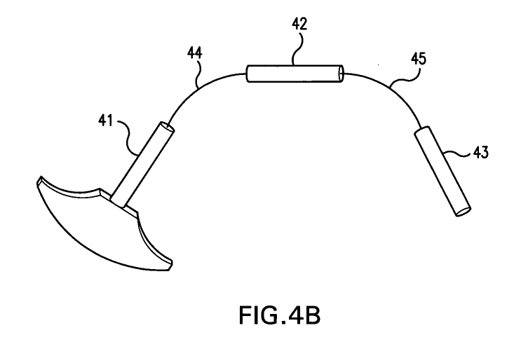

As illustrated in FIG. 3, in an embodiment, the individual segments 41, 42, 43 may be connected to each other by connectors 44, 45. In an embodiment, the connectors may comprise a material that is substantially rigid. The connectors may also comprise portions that are substantially rigid combined with portions that are bendable. For example, the connectors may allow for the device to bend at the point where the connector element joins to the segment. FIG. 4A illustrates an embodiment where the segmented plunger comprises a bend at the distal end 44b of connector 44 and the proximal end 45a of connector 45.

Rather than being rigid, the connector(s) may comprise a material that is substantially flexible, such that the connector itself is able to bend. FIG. 4B illustrates an embodiment where the device comprises bending within connectors 44 and 45. For example, the segments may be connected to each other by a flexible connector such as a bendable wire. Stainless steel may be used to form the connectors as stainless steel is strong and relatively inexpensive.

In another embodiment, the connector may comprise a material that comprises structural or shape “memory.” In an embodiment, a shape memory material, such as nitinol, may be used. As is known in the art, a shape memory material may be urged from a first shape to a second shape by the application of external energy, but when the external energy is removed, the material will resume its original shape without loss of strength or internal structure. For example, one can bend a straight wire that is made of a shape memory alloy, and upon removing the force required to bend the wire, the wire will resume its straight conformation. As is known in the art, such shape memory materials are commercially available in various compositions, conformations, surface finishes, transformation temperatures, and the like, which can be selected to optimize the performance characteristics required. Nitinol is a commonly used shape memory alloy containing almost equal parts of titanium and nickel. Nitinol may, in certain embodiments, recover from significantly greater deformation compared to most other shape memory alloys.

The material may further comprise a temperature-sensitive shape memory material such that exposure of the connector to the heat of the subject's body may result in the connector being able to assume a second conformation different than a first conformation. For example, nitinol is also commonly used biomaterial with thermal shape memory properties. A connector made from a temperature-sensitive shape memory alloy can be deformed (e.g., bent) to a shape suitable for insertion into an access member under conditions of limited clearance, with a thermally-induced reversal of the deformation (e.g., from bent to straight) when the connector element is threaded though the cannula. The applied heat can be from the surrounding tissue, or may be externally applied. Temperature-sensitive shape memory alloys are available in a wide range of transformation temperatures appropriate for the clinical setting, including those alloys (such as nitinol) that exhibit a transformation temperature at body temperature.

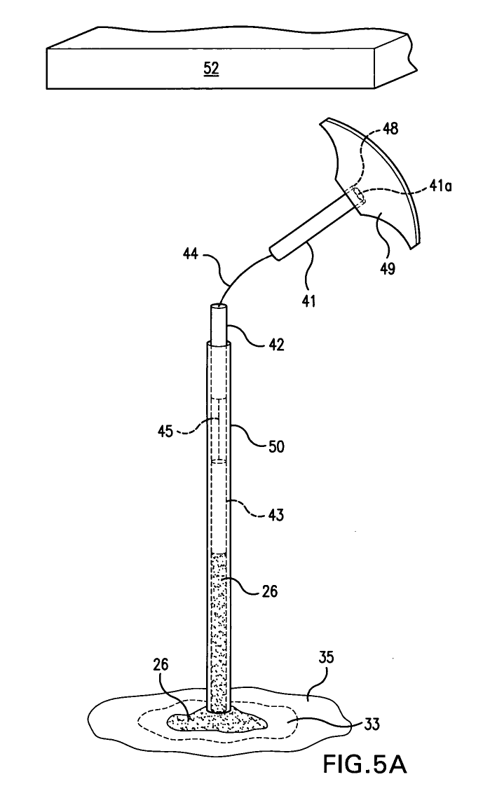

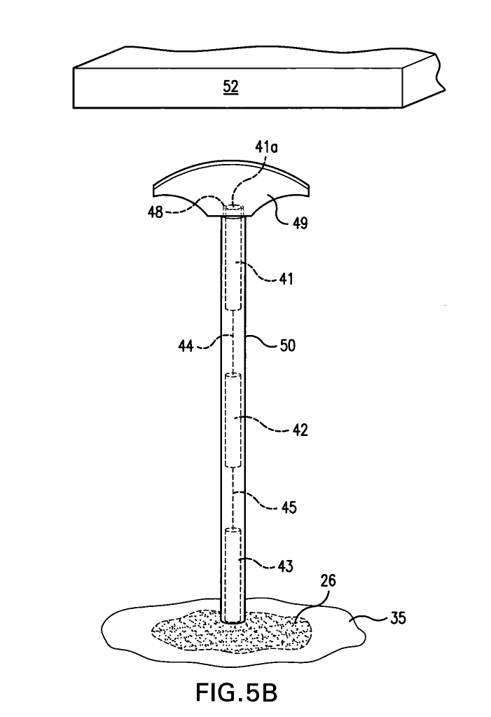

As described herein, the segments may be employed to urge a material through a cylindrical tube, such as a delivery cannula, for delivery of the material to a body part. In an embodiment, the connectors maintain a straight conformation as the plunger is urged through a cylindrical tube. FIGS. 5A and 5B illustrate a segmented plunger of the present invention being inserted into an access member comprising a tube 50, and being used to urge a material 26 through the tube 50. It can be seen that while the proximal connector 44 can be bent to allow access to the tube 50, the distal connector 45 maintains a straight conformation once any force to bend the connector is removed (FIG. 5A). Similarly, FIG. 5B shows the segmented plunger being pushed through the tube 50 using handle 49. Thus, because the tube has a straight conformation, the connectors will maintain a substantially straight conformation as the segmented plunger is urged through the tube 50. In an embodiment, the tube 50 may comprise the hollow outer cylinder of a bone filler device (handle not shown in FIGS. 5A and 5B). As illustrated in FIGS. 5A and 5B, in one embodiment, the predetermined location of interest in the subject may comprise a bone 35 having a cavity 33 for emplacement of a bone filler material 26.

In an embodiment, the product of the present invention may comprise at least one tool for urging the plurality of segments through an access member for delivery of a material to an internal body part in a living body. In an embodiment, the segmented plunger may comprise a handle 49 as a tool to push the plurality of segments through an access member. The handle may fit on the proximal end of at least one of the segments to allow an individual to urge the segment through a tubular access member 50 by pushing on the handle (FIG. 5B). For example, the handle 49 may be positioned on the proximal end 41 a of the proximal-most segment 41 of the device 40. The handle 49 may comprise a recessed portion 48 to engage the segment (FIGS. 5A and 5B). The handle may be permanently attached to the segment, or it may be removable.

In some cases, as shown in FIG. 5A, where there is restricted clearance due to positioning of a C-arm or other imaging device 52, it may be necessary to bend the plunger as the segments are being inserted into a tubular access member 50. Bending the proximal connector 44, however, can effectively prevent using the handle 49 to push the segmented plunger through the tube as the handle 49 and the tube 50 are not aligned. Thus, in an embodiment, the device may comprise an additional tool for urging distal segments of the plunger through the tube.

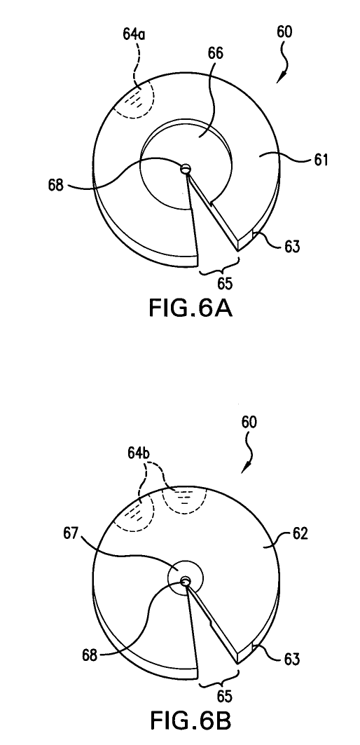

An embodiment of a disk-shaped tool that may be used for urging segments of a segmented plunger through a tube is shown in FIGS. 6A and 6B. As described herein, the tool may comprise various shapes depending upon the specific application for which the product is being used. Also, the tool may be configured to be in communication with an end of at least one of the plurality of segments. Thus, in one embodiment, the tool 60 for urging segments through an access member may comprise a distal surface 61 designed to engage the proximal end of the segments as they are urged through an access member. FIG. 6A shows an embodiment of a distal (i.e., positioned farthest from the user) surface 61 of a disk-shaped tool of the present invention, and FIG. 6B shows an embodiment of a proximal (i.e., positioned closest to the user) surface 62 of a tool of the present invention. The tool for urging the segments of a segmented plunger through an access member may comprise a variety of shapes, but generally will comprise a size that is able to easily fit between connected segments (e.g., 41, 42, and 43 as illustrated in FIGS. 5A and 5B) of the plunger and that allows access in the space provided by the C-arm or other imaging device. Thus, the tool may be substantially planar in that the tool is restricted in height 63 (FIG. 6A) with respect to the z-axis of three dimensional space, but may be less restricted in size in the x and y dimensions, where the z-axis comprises the linear axis of the access member 50 through which the individual plunger segments, e.g., 41, 42, and 43, are being pushed.

In certain embodiments, the tool 60 for urging the segments through an access member, such as a tube, may comprise a means for a user to manually manipulate the tool. Thus, in an embodiment, the tool may comprise a handle. Or, the tool 60 may comprise finger grips 64a, 64b, on each surface of the tool (FIGS. 6A and 6B) by which a user may grab the tool. To allow for the tool to be juxtaposed against the end of the segment that is being pushed through an access member, the tool may comprise an aperture 65 to allow the tool to wrap around the connector segment extending from the access member. The aperture 65 may extend from the outer perimeter of the tool to the center of the tool.

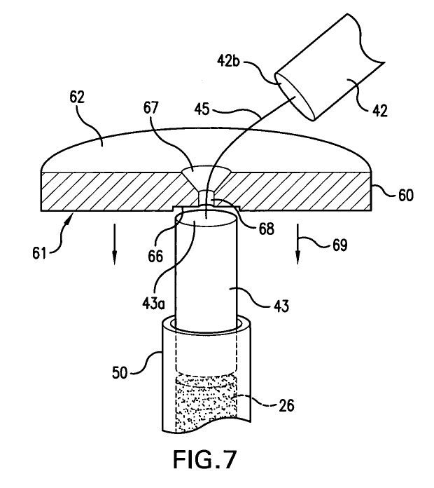

FIG. 7 illustrates one embodiment of how a tool 60 of the present invention may be used to push a segmented plunger comprising connected segments 42 and 43 through a tubular access member 50 comprising a material 26 to be emplaced in a body part. In FIG. 7, the direction of pushing is shown by the arrows 69. In an embodiment, the connector 45 may threaded through the slit 65 (FIG. 6) of the tool such that the distal surface 61 of the tool 60 is able to abut the proximal end 43a of one of the segments 43 (FIG. 7). FIG. 7 further illustrates that the tool 60 may also comprise a means for engaging the segments. Thus, the tool may be configured to be in communication with an end of at least one of the plurality of segments. For example, in an embodiment, there may be a recessed surface 66 in the tool 60 that is shaped to engage the flat surface 43a of the segment extending from the tubular access member 50.

In an embodiment, the tool for pushing segments through an access member such as a tube may comprise a means to allow the connectors that are outside of the access member to be bent as segments that have been inserted into the access member are pushed towards the body location of interest. For example, as illustrated in FIGS. 6A, 6B, and 7, the tool may comprise a funnel-shaped opening 67 that facilitates gentle bending of the connector element. The funnel-shaped opening may narrow at the bottom 68 to allow the connectors to straighten as they enter the tubular access member 50.

The tool for pushing segments through an access member may, in certain embodiments, be made of a material that is compatible with the other parts of the system. For example, the tool may be made of metal such as aluminum, stainless steel, spring steel, a nickel titanium alloy or other alloys. Or, the tool may be made of plastic, such as polypropylene, polyethylene, polyethyleneteraphthalate (PET), ionomer, polycarbonate or nylon. One of ordinary skill in the art having the benefit of this disclosure would appreciate that other materials, including those that are well-known to one in the art, may be applied to configure the segments described herein. In one embodiment, a pushing tool 60 may be used to urge each of the segments through a tube such that an additional handle is not required.

As described above, in certain embodiments, the segments may be connected to each other so as to comprise a single device having a plurality of segments. The number of segments may be varied depending upon the size of the outer cylinder. For example, in alternate embodiments, where the product comprises a plunger for a bone filler device, a cannula for accessing the bone may comprise dimensions on the order of about 2 to 20 inches (50.8 millimeters (mm) to 508 mm), or about 4 to 15 inches (101 mm to 381 mm), or about 6 to 12 inches (152 mm to 305 mm), or about 8 inches (203 mm) in length. Also, in alternate embodiments, the cannula may range from about 0.05 to 0.5 inches (1.27 mm to 12.7 mm), or from about 0.1 to 0.3 inches (2.5 mm to 7.6 mm), or from about 0.12 to about 0.2 inches (3.1 mm to 5.1 mm), or from about 0.14 to 0.16 inches (3.6 mm to 4.1 mm) in diameter. Thus, the segmented plunger may comprise a total length that is about the same as the cannula, with a slightly smaller diameter, e.g., about 0.04 to 0.45 inches (1.0 mm to 11.4 mm), or from about 0.09 to about 0.29 inches (2.3 mm to 7.4 mm), or from about 0.1 to about 0.19 inches (2.5 mm to 4.8 mm), or from about 0.13 to 0.15 inches (3.3 mm to 3.8 mm).

Also, the number of segments may vary depending upon the particular application. For use as a plunger for a bone filler device, the device may comprise, in alternate embodiments, from about 2 to about 20 segments, or from about 2 to about 10 segments, or from about 2 to about 5 segments, or 2 to 4 segments, or 2 to 3 segments. Where the segments are connected, the device may comprise N-1 connectors, where N equals the number of segments. Thus, in alternate embodiments, for a plunger having 5 segments there may be 4 connectors, and for a plunger having 3 segments there may be 2 connectors, and so forth.

The use of connectors allows for the plurality segments to be provided as a single part. Alternatively, the plurality of segments may be provided as individual parts that are not physically connected to each other. Thus, in one embodiment, the product of the present invention may comprise a plurality of segments designed to urge a material through a tube, wherein the segments are not physically connected to each other.

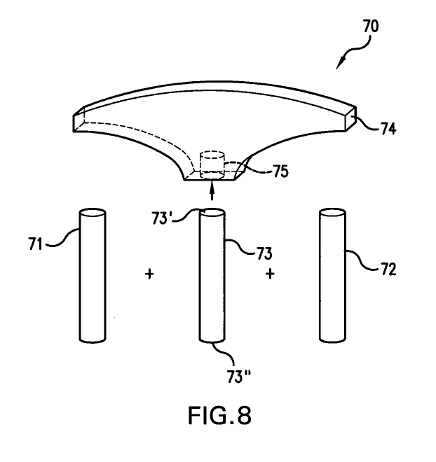

FIG. 8 shows an example of a segmented plunger comprising a plurality of segments, where the segments are not connected to each other. In one embodiment, the product may comprise a plurality of segments (e.g., 71, 72, 73), and a means for the user to engage the segments to thereby urge the segments through an access member to deliver of a material to a location in a subject. In one embodiment, the means for the user to engage the segments may comprise a handle 74. Alternatively, a substantially planar tool 60 as described above for pushing the segments through the access member may be used. The handle or tool may comprise a fitting or recessed portion, e.g., 75 of handle 74 (or 68 of the pushing tool 60 as shown in FIG. 7) into which the proximal end of segments 71, 72, and 73 may be inserted. In an embodiment, the segments may be cylindrical in shape. In an embodiment, the segments may comprise substantially identical surfaces at each end such that either end (e.g., 73′ or 73″) may eventually become either the proximal or the distal end of the segment (FIG. 8). Similar to a plunger with connected segments, the number of segments may vary depending upon the particular application. Thus, the product may comprise, in alternate embodiments, from about 2 to about 20 segments, or from about 2 to about 10 segments, or from about 2 to 5 segments, or from 2 to 4 segments, or 2 to 3 segments.

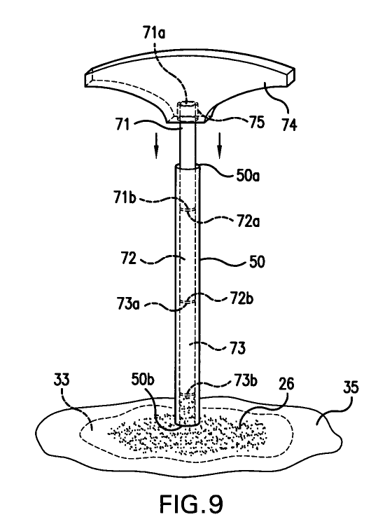

FIG. 9 illustrates an embodiment of the use of a product of the present invention comprising separate segments 71, 72, and 73 for urging a material 26 through a tubular access member 50 for delivery of the material to a body part of interest. For example, the distal end 73b of segment 73 may be inserted into the proximal end 50a of a tube that has been loaded with a material 26 to be emplaced in a body part. Once the first segment is at least partly inserted in the tube 50, the handle 74, or a pushing tool 60 as described above, may be used to engage the proximal end of the cylindrical segment 73a that extends from the tube 50. Alternatively, the handle 74 may be fitted onto the one end of the segment 73 prior to inserting the segment 73 into the tube 50. Once the first segment (e.g., 73) has been pushed through the tube 50, the handle 74 (or pushing tool 60) may be disengaged, and the second segment 72 inserted into the handle. The second segment 72 may then be inserted in the tube 50 such that the distal end 72b of the second cylinder 72 abuts the proximal end 73a of the first cylinder 73. The second segment may then be pushed through the tube until the proximal end 72a of the second segment is substantially aligned with the proximal end 50a of the tube. The handle 74 may again be disengaged, and a third segment 71 inserted. The third segment 71 may then be inserted in the tube 50 such that the distal end 71b of the third segment 71 abuts the proximal end 72a of the second segment 72. The third segment may then be pushed through the tube until the proximal end 71a of the third segment is substantially aligned with the proximal end 50a of the tube. This process may be repeated with additional segments as needed until substantially all of the material 26 that was loaded in tube 50 has been deposited at the body part of interest. For example, in one embodiment, the body part of interest may comprise a bone 35 having a cavity 33 for emplacement of a bone filler material.

Also, in an embodiment, the segments may comprise a means to have adjacent segments interlock. For example, in one embodiment, the end of one segment may be tapered so as to fit within a recessed opening in the end of another segment. For example, the proximal end of segment 72 (e.g., 72a) may be tapered so that it can be inserted into an overhanging lip that may be molded into the distal end of the next segment (e.g., 71b).

Other embodiments of the present invention comprise kits and/or systems for treatment of internal body parts. Thus in one embodiment, the present invention may comprise a system for delivering a material to a predetermined location in a subject. In an embodiment, the system may comprise an access member. In one embodiment, the access member may comprise a path for delivering a material to a predetermined location within a subject. Also, in an embodiment, the system may comprise a segmental plunger for delivering the material to the predetermined location. The plunger may, in certain embodiments, comprise a plurality of segments each having a diameter that is less than the internal diameter of the access member and a length that is less than the length of the access member. For example, in one embodiment, the present invention provides a system for delivering a material to a predetermined location in a subject comprising: (a) an access member; and (b) a plunger having a plurality of segments each having a diameter less than the internal diameter of the access member and a length that is less than the length of the access member.

In other embodiments, the present invention may comprise a surgical or medical kit. The kit may be suitable for medical or veterinary use, as for example, for emplacement of a material in a human, or an animal. For example, in an embodiment, the present invention may comprise a surgical or medical kit comprising a device delivering a material to a predetermined location in a subject. In one embodiment, the kit may comprise: (a) an access member; (b) a plunger having a plurality of segments each having a diameter less than the internal diameter of the access member and a length that is less than the length of the access member; and (c) instructions for use. In one embodiment, the access member may comprise a path for delivering the material to the predetermined location.

The subject for which the systems and kits of the present invention may be employed may comprise an animal. For example, the subject may comprise a mammal. In one embodiment, the subject may be a human. The user of the product requiring access to the subject may be a physician, veterinarian, or a health care professional (e.g., physician's assistant, nurse, or technician). In alternate embodiments, however, a user of the systems and/or kits of the present invention may be accessing a particular location in his or her own body, as for example, for periodic delivery of a therapeutic material.

The predetermined location may, in certain embodiments of the systems and kits of the present invention, comprise a body part within a living body. In an embodiment, the predetermined location may comprise a bone. In one embodiment, the predetermined location may comprise a bone interior. For example, the predetermined location may comprise a portion of a spine. Thus, in one embodiment, the plunger may be sized for urging a material through an access member to thereby deliver the material to a bone interior, such as a vertebral body or disc of a spine.

For example, as described herein, due to various traumatic or pathologic conditions, such as osteoporosis, a vertebral body can experience a vertebral compression fracture (VCF). Thus, the systems and kits of the present invention may by used to repair a vertebral body lost due to a fracture, or when other degeneration occurs. The systems and kits of the present invention may also be used to repair other parts of a living or non-living organism. For example, in certain embodiments, the kits and systems of the present invention can be deployed in other bone types and within or adjacent other tissue types, such as in a vertebral disc, an arm bone, a leg bone, a knee joint, or the like.

The access member used with the systems and kits of the present invention may provide a path to access a region or a body part that is located within a subject's body. The access member may be any type of device that can extend from the location of interest (e.g., a bone or an organ) to be accessible to a user of the access member. For example, the access member may be designed to extend from an internal body part in a subject to outside of the subject's body. The access member may comprise an elongated hollow member such as a hollow cylinder or a tube. Thus, in an embodiment, the tube may be designed to provide an access from outside of the living body to the internal body part. In an embodiment, the segments of the plunger and the access member are substantially cylindrical in shape. For example, the access member may comprise a cannula, such as a cannula used to deliver a material to bone or another type of body part.

In certain embodiments, the access member of the systems and kits of the present invention may be configured to provide percutaneous surgical access from outside of the subject to the predetermined location. In alternate embodiments, the percutaneous surgical access may comprises an incision ranging from about 0.1 to 4.0 centimeters (cm) in diameter, or from about 0.2 to 2.0 cm in diameter, or from about 0.25 to about 1 cm in diameter. Thus, in alternate embodiments, the percutaneous surgical access may comprise an incision that is less than 4 cm in diameter, or less than 2 cm in diameter, or less than 1 cm in diameter. In one example embodiment, the percutaneous surgical access may comprise an incision of about 1 cm in diameter. For example, in a typical percutaneous surgical repair of a spine, a cannula may establish a percutaneous path along its elongated axis to a vertebral body of one of the several vertebrae.

In some embodiments, each of the plunger segments used with the systems and/or kits of the present invention comprises a diameter less than the internal diameter of the access member and a length less than the length of the access member. In this way, the plurality of segments may be inserted into the access member and function to push a material that has been loaded in the access member from one end of the access member to the other end of the access member. Also, in some embodiments, the plurality of the segments together comprise a length that is less than, or substantially the same as, the length of the access member. For example, a plunger comprising three segments may have an overall length that is less than, or substantially the same as, the length of a tube comprising a material for delivery to a body part. In this way, the plunger will not extend out the distal end of the tube after the plunger has been used to urge the material in the tube out the distal end of the tube for delivery to a body part.

The plurality of segments used in the plunger of the systems and kits of the present invention may be connected as one unit, or may be separate parts. Thus, in an embodiment, at least two of the segments are attached to each other by a connector. The connector may be fixedly or reversibly connected to the segments. The connector may, in certain embodiments, comprise a material that is bendable. This may allow the plunger to assume various conformations as the segments are urged through the tube. Also, in an embodiment, the connector may comprise a material having shape memory. By having shape memory, a connector having a first conformation may be urged into a second conformation, and then regain its original (first) conformation as the plunger is urged through the tube. In an embodiment, the changes in conformation of the material having shape memory are induced by changes in temperature as described herein. Additionally or alternatively, it may be important for the connector to provide structural strength and to maintain an overall linear conformation as the plunger urges the material for delivery to the body part through the tube. Thus, in an embodiment, the connector may comprise a substantially rigid material. In an embodiment, the connector is substantially straight.

In certain embodiments, the plunger of the systems and kits of the present invention may comprise a plurality of segments sized for urging a material through an access member for delivery of a material to a predetermined location in a subject, where the segments are not connected to each other. For example, in some embodiments, the plurality of segments may be configured such that an end of a first segment can be positioned to be in direct communication with an end of an adjacent segment.

In certain embodiments, the segmented plunger systems and kits of the present invention may provide a means to deliver a material to a predetermined location in a subject, such as an internal body part, where the working environment comprises limited access. Thus, embodiments of the systems and kits of the present invention may further comprise at least one tool for urging at least one of the segments through the access member. In an embodiment, the segment being urged through the access member travels from the proximal end of the access member (i.e., the end closest to the user) towards the distal end of the access member (i.e., the end farthest from the user). The tool of the present invention may, in certain embodiments, comprise a size and shape to accommodate limited access to the subject being treated.

The tool may be used to urge the segments through the access member either individually, or as a linear unit of segments. In an embodiment, the tool may be configured to be in communication with an end of at least one of the plurality of segments. For example, the tool may be shaped to engage the proximal end of at least one of the plurality of segments. In alternate embodiments, the tool may either be fixedly attached to the segment, or the tool may be removably attached to the segment.

The tool used with the systems and kits of the present invention may comprise various shapes depending upon the specific application for which the product of the present invention is being used. Also, in certain embodiments, a variety of different tools for urging at least one of the segments through the tube may be used. In one example embodiment, the tool may comprise substantially flat surface configured to engage a surface of one of the segments. For example, the tool may configured to engage the distal end of a segment that extends from the proximal end of the access member to push the segment towards the distal end of the access member. Alternatively, the tool may comprise a configuration designed to pull segments that are outside of an access member into the access member.

In an embodiment, the tool may comprise a handle. Alternatively or additionally, the tool may comprise a substantially planar conformation. In an alternate embodiments, the tool may comprise a substantially planar conformation that is rectangular, polygonal, circular, or oval in shape. In an embodiment, the tool comprises a disk-like shape.

In an embodiment, the tool used in the systems and/or kits of the present invention may comprise a means for the tool to be positioned around a connector connecting a first segment to a second segment. For example, the tool may comprise an aperture that allows the tool to be positioned around a connector connecting two segments together. The aperture may comprise a variety of shapes. In an embodiment, a portion of the aperture comprises a substantially circular shape. The aperture may be designed to have a larger opening on the proximal surface of the tool than on the distal surface of the tool. For example, as discussed herein, a portion of the aperture may comprise a funnel-like shape having a wide opening to allow for bending of the connector that extends from the access member, but having a straight portion to align the distal end of the connector with the access member.

The tool may be shaped to allow for a user of a system and/or kit of the present invention to easily manipulate the tool. Thus, the tool may comprise a handle, or a surface shaped to engage at least a portion of a user's hand. For example, the tool may comprise indented portions shaped to facilitate grasping the tool with one's hand.

In an embodiment, the kit may further comprise a container for holding each of the parts under sterile conditions, or for transporting the parts from a first site to a second site. The kit may, in some embodiments, comprise a tray for holding the various parts in a secure position during sterilization and/or transport. In an embodiment, the parts of the kit are arranged in an organized layout to facilitate use of the segmented plunger and other components of the kit for delivery of material to an internal body part. Also, labels identifying various parts of the kit may be included.

In an embodiment, the kit may comprise an inner seal, comprising an inner wrap, that may be sealed by heat or vacuum, to prevent the components of the kit from being exposed to the outside environment. The inner seal may comprise a conventional peal-away seal to provide quick access to the components of the kit. Also, in an embodiment, the kit may include an outer wrap, also sealed by heat or the like, to enclose the inner wrap. Like the inner seal, the outer seal may comprise a conventional peal-away seal to provide quick access to the components of the kit. Use of an outer wrap may allow the kit to be prepared for imminent use by removing the outer wrap while leaving the inner wrap in place to ensure sterility of the kit components. The kit may also comprise a case to protect the components of the system from physical damage. In an embodiment, the outer wrap may be made of materials commonly used in the art such as polyethylene and MYLAR™, to allow for visualization of the components in the kit. The inner wrap may be made of materials such as TYVEK™ (DUPONT®), that is permeable to ethylene oxide (ETO) sterilizing gas. Sterilization may be by heat, pressure and/or sterilization gas as is known in the art. Also, the kit may include directions for use by a physician or other trained personnel.

Embodiments of the system and/or kits of the present invention may further comprise a material to be delivered to the internal body part. In an embodiment, the material to be delivered to the body part may be emplaced within at least a portion of at least one access member. For example, where the system is being used for bone repair, a tube may be loaded with a bone filler material or an adhesive. In an embodiment, the bone filler material may comprise a mixture containing calcium, hydroxyl apatite, and a polymer. Also, in certain embodiments, the bone filler may comprise ceramic granules or other filler material.

In other embodiments, the material may comprise an autograft or allograft bone graft tissue (see e.g., Dick, Archives of Orthopaedic and Traumatic Surgery (1986), 105: 235-238; or Bhan et al, International Orthopaedics (SICOT) (1993) 17: 310-312). The bone graft tissue can be obtained using a Bone Graft Harvester, which is commercially available from SpineTech. Alternatively, the material may also comprise a granular bone material harvested from coral, e.g., PROOSTEON™ calcium carbonate granules, available from Interpore. The granules may be loaded into the access member using a funnel or other loading means. The material for delivery to a bone can also comprise demineralized bone matrix suspended in glycerol (e.g., GRAFTON™ allograft material available from Osteotech), or SRS™ calcium phosphate cement available from Novian. The material for delivery to a bone can also be in sheet form, e.g., COLLAGRAFT™ material made from calcium carbonate powder and collagen from bovine bone. In an embodiment, the sheet may be rolled into a tube and loaded by hand into the access member. In one embodiment, KYPHX®HV-R™ bone cement, commercially available from Kyphon, Inc., may be used.

The system and/or kits of the present invention may comprise one, or a plurality, of access members (e.g., cannulas) that are loaded with material that is to be delivered to a predetermined location in a subject, such as a body part or region. Thus, in one embodiment, after the material in one of the access members has been dispensed to the body part of interest, the first access member and segmented plunger may be removed and an additional access member filled with material to be emplaced may be positioned for delivery of a second aliquot of the material. At this point, an additional plunger may be inserted into the newly placed access member. Or, the segmented plunger used to deliver the first aliquot of the material may be removed from the first (e.g., spent) access member and used to deliver a second aliquot of material from the second access member.

The access members (e.g., tubes or cannulas), and plungers comprising a plurality of segments, may vary in size depending upon the body part to be accessed. In one embodiment, the systems and/or kits of the present invention may comprise a bone filler device. The bone filler device may range in size depending upon the bone to be repaired. For example, the access member and/plungers may be sized to fit pre-existing bone filler devices such as the KYPHX®EXPRESS™ bone filler device that is commercially available from Kyphon, Inc. For example, to repair a single thoracic vertebra may require up to about 12 cubic centimeters (cc) of bone filler material. Using a cannula that is about 8 inches (203 mm) long and about 0.137 inches (3.5 mm) wide may require about 6 to 8 cannulas of bone filler material. More or less material may also be delivered depending upon the size of the cavity to be filled. For example, where a cavity is created by drilling a hole in a bone and compacting any bony material with an expandable device such as a balloon, the amount of material required for a specific application may be determined by monitoring the inflation volume of the balloon.

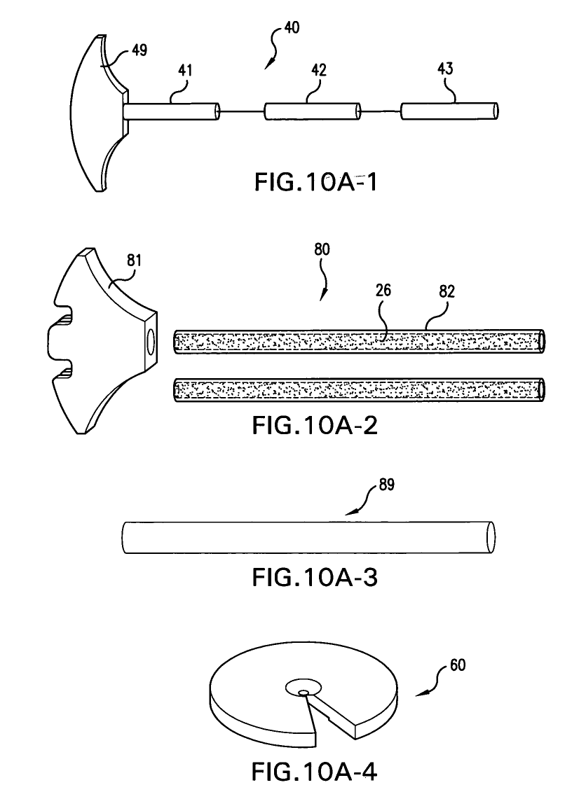

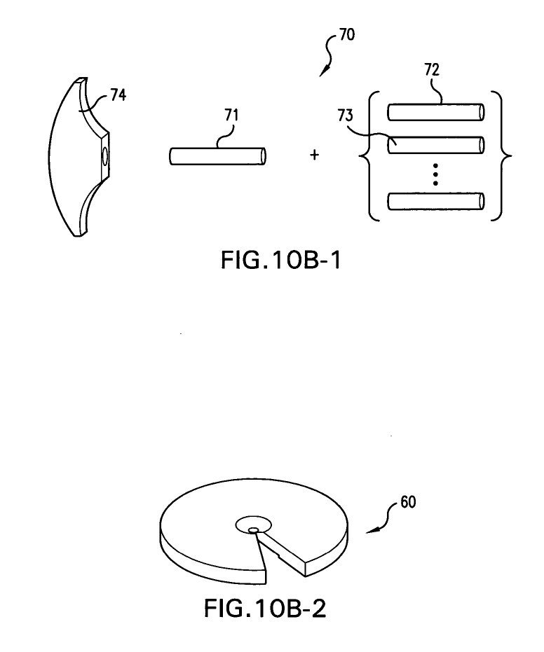

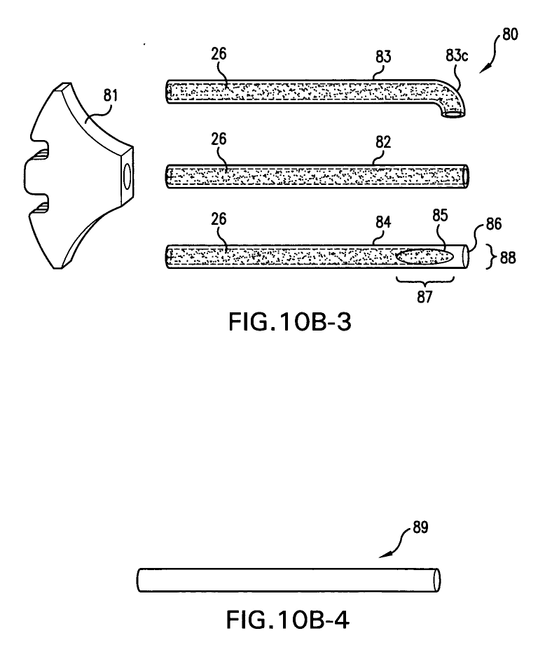

An example of an embodiment of a system of the present invention where the segmented plunger comprises connected segments is shown as FIG. 10, panels A1-A4. Thus, the system may comprise a plunger 40 comprising a plurality of connected segments 41, 42 and 43. The plunger segments 41, 42, and 43, may each have a diameter less than the internal diameter of the access member and a length less than the length of the access member that will be used to access the location of interest in the subject. In an embodiment, the plunger 40 may comprise a handle 49. The handle may be fixedly attached or removably attached to one of the segments of the plunger. The system may further comprise a plurality of access members 80 each comprising a tube 82 designed to extend from a predetermined location in the subject, such as an internal body part, to outside of the subject so as to provide access to the body part. Also, in some embodiments, the access member 80 may comprise a handle 81. Also, in an embodiment, multiple access members may be used.

Although a handle 49 may be used to urge the plurality of segments through the tube 82 for delivery of a material to a predetermined location in a subject's body, a tool 60 (FIG. 10A-4) for pushing the segments through the access member may be included with the system of the present invention. The tool for pushing segments through the access member may be used as described above.

In addition to the access member 82 used to deliver a material to the internal body part, the system may comprise a second member comprising a means to access the body part of interest. In an embodiment, the second access member 89 may comprise a hollow cylinder or tube. The second access member may comprise a diameter that is greater than the diameter of the first access member 80. For example, the second access member 89 may comprise an outer cannula that may be used to provide a drill bit or other cutting tool for cutting into a bone requiring repair. Once the interior of the bone has been accessed, a balloon catheter may be inserted into the second access member 89 and threaded to the site of the bone requiring repair. Inflation of the balloon may then be used to compact any deteriorated bone tissue, and to create a cavity in the bone. Thus, alternate embodiments the system may also comprise a cutting tool (not shown) and a balloon catheter (not shown) similar to those in the art used for the repair of bone.

In other embodiments, the system may comprise a segmented plunger having segments that are not connected to each other. FIG. 10, panels B1-B4, shows an illustration of various embodiments of such a system. Thus, the system may comprise separate segments, e.g., 71, 72, and 73 (and in some cases additional segments), as the plunger (FIG. 10B-1). The plunger segments 71, 72, 73, may each have a diameter less than the internal diameter of the access member and a length less than the length of the access member that will be used to access the location of interest in the subject.

The system utilizing separate plunger segments may further comprise a plurality of access members 80 (FIG. 10B-3). In an embodiment, the access members may each comprise a tube designed to extend from a predetermined location in the subject, such as an internal body part, to outside of the subject so as to provide access to the body part. In some embodiments, the access members 80 may comprise a handle 81. Also, in an embodiment, multiple access members may be used. Similar to the system using a plunger comprising connected segments, the system may comprise a plurality of access members 80 each comprising a tube designed to extend from outside of a living body to an internal body part.

Similar to a system using a plunger with connected segments, the unconnected segments may comprise a handle 74 (FIG. 10B-1), and/or a substantially planar tool 60 (FIG. 10B-2) to push the segments through the access member(s). For example, where the system comprises a handle 81 (FIG. 10B-3) as part of the access member 80, an inner handle 74 may be used to push the segments through the access member.

Where the segments of the plunger are not connected to each other, it may be beneficial to have some means to prevent the most distal segment from exiting the access member, and thus, being expelled into the body part being treated. In one embodiment, the individual segments of the plunger are of a length such that a plurality of the segments, when aligned, together comprise substantially the same length as the length of a linear access member 82 used for delivery of a material to the body part. In this way, when the plurality of segments are urged through the access member, the most distal segment will be aligned with the distal end of the access member but will not exit out of the distal end access member.

In another embodiment, the access member for delivery of the material to a body part may comprise a non-linear shape (FIG. 10B-3). Using a non-linear access member may restrict the segments from exiting the distal end of the member. For example, the access member 83 for delivery of a material to a body part may comprise a tube having a bent portion 83c at the distal end of the tube to prevent the most distal segment from exiting the tube (FIG. 10B-3). Or, the access member 84 may comprise a tube having a side port 85 for expulsion of the material 26 to the internal body part (FIG. 10B-3). Where the tube comprises a side port 85, the distal end 86 of the tube may be sealed. In an embodiment, the side port comprises a shape such that the length 87 of the opening is greater than the diameter 88 of the tube (FIG. 10B-3), so as to allow the material to exit the tube without any build up of pressure at the distal end of the tube.

Also, in an embodiment, the system may comprise a second access members 89 allowing for emplacement of the tubular access members at the predetermined location of interest (FIG. 10B-4). The second access member may comprise a diameter that is greater than the diameter of the first access member 80. For example, the second access member 89 may comprise an outer cannula that may be used to provide a drill bit or other cutting tool for cutting into a bone requiring repair. Once the interior of the bone has been accessed, a balloon catheter may be inserted into the second access member 89 and used to compact any deteriorated bone tissue, and to create a cavity in the bone. Thus, as described for a system having a plunger with connected segements, alternate embodiments of the system may also comprise a cutting tool (not shown) and a balloon catheter (not shown) similar to those in the art used for the repair of bone.

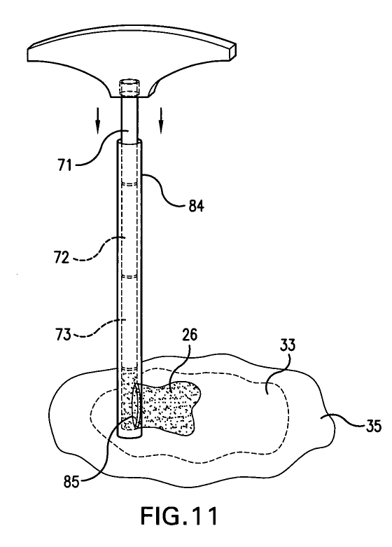

FIG. 11 shows an embodiment of a system of the present invention comprising a segmented plunger having a plurality of unconnected segments and an access member comprising side port being used to deliver a material to a bone interior. Thus, it may be seen that as the most distal segment 73 engages the material 26 to be delivered to a body part 33, the material exits the access member 84 via the side port 85.

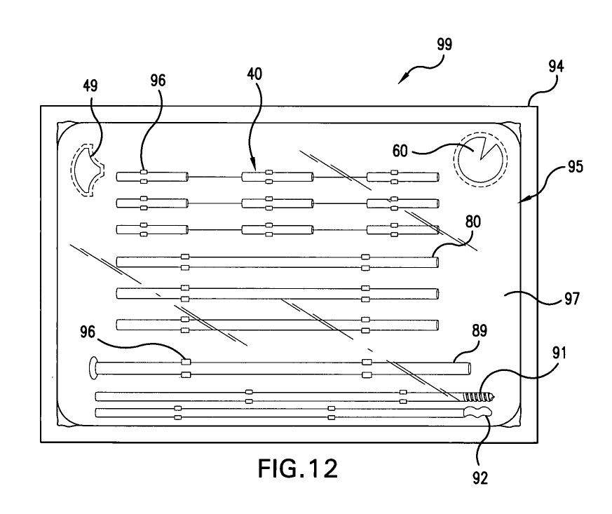

FIG. 12 shows an embodiment of a kit 99 of the present invention. In an embodiment, the kit may comprise a container 94 for holding each of the parts under sterile conditions, or for transporting the parts from a first site to a second site. The kit may comprise a plurality of segmented plungers 40 and access members 80. In an embodiment, the access members may be filled with a material to be delivered to a body part of interest. Also, the kit may comprise a handle 49 or tool 60 for pushing the segments through the delivery cannula. Also, an outer access member 89, drill bit 91, balloon catheter 92, and other components may be included. The kit may comprise a tray 95 comprising clips 96 or other fastening means for holding the various parts in a secure position during sterilization and/or transport. In an embodiment, the parts of the kit are arranged in an organized layout to facilitate use of the segmented plunger and the access member(s) for delivery of material to an internal body part. Also, labels identifying various parts of the kit may be included.

In an embodiment, the kit may comprise an inner wrap 97, that may be sealed by heat or vacuum to prevent the components of the kit from being exposed to the outside environment. Also, in an embodiment, the kit may include an outer wrap (not shown) and/or a case 94 to protect the components of the system from physical damage. The inner/outer wraps may be made of wrap materials commonly used in the art such as polyethylene, TYVEK™, or MYLAR™, to allow for visualization of the components in the kit and or sterilization using a sterilizing gas. Sterilization may be by heat, pressure and/or sterilization gas as is known in the art. Also, the kit may include directions for use by a physician or other trained personnel.

Embodiments of the present invention may also comprise methods for delivery of a material to an internal body part. For example, in one embodiment, the method may comprise a method for delivery of a material to a predetermined location in a subject comprising the steps of: inserting an end of a first segment of a plunger comprising a plurality of segments into the end of an access member comprising at least a portion of the material to be delivered, the segments each comprising a diameter less than the internal diameter of the access member, and urging the segment at least partially through the access member. The access member may be loaded with an amount of the material for delivery to the predetermined location as is required. Thus, in alternate embodiments, the access member may comprise an amount of material that is less than, about the same as, or greater than, the amount of material that is ultimately to be delivered to the location of interest. Thus, in an embodiment, the method may further comprise the step of loading the access member with at least a portion of the material to be delivered to the predetermined location in a subject. As described above, the access member may provide access to the predetermined location for person performing the method. Thus, the method may further comprise the step of positioning the access member such that one end of the access member is located at the predetermined location and the other end of the access member is accessible to a user.

Using the segmented plunger of the present invention may provide flexibility with respect to the amount of material delivered to the predetermined location, depending upon the total number of segments that are urged through the plunger. Thus, in one embodiment, the method may comprise the step of inserting an end of an additional segment of the plurality of segments into the end of the access member, and urging the additional segment through the access member. Thus, in an embodiment, the plurality of the segments together comprises a length that is less than, or substantially the same as, the length of the access member.

For example, in an embodiment, the method may comprise delivering a bone cement or filler material to a bone requiring repair. The method may comprise, in certain embodiments, the step of loading an access member comprising a path for delivery of the material to the predetermined location with at least a portion of the material. The method may further comprise positioning the access member such that one end of the access member is located at the predetermined location, and the other end of the access member is accessible to a user. The method may further comprise inserting an end of a first segment of a plunger comprising a plurality of segments into the end of the access member that is farthest from the predetermined location. The method may also comprise urging the first segment towards the end of the access member that is positioned at the predetermined location in the subject to thereby urge the material loaded in the access member towards the predetermined location. Also, the method may comprise the subsequent steps of inserting an end of an additional segment of the plurality of segments into the end of the access member that is farthest from the predetermined location, and urging the additional segment towards the end of the access member positioned at the predetermined location to thereby urge the material loaded in the access member towards the predetermined location in the subject. The step of pushing additional segments through the access member may be repeated until the correct amount of material is delivered to the predetermined location in the subject.

Thus, in one example embodiment, the method may comprise the steps of: loading an access member with at least a portion of a material to be delivered to a predetermined location in a subject, the access member comprising a path for delivery of the material to the predetermined location; positioning the access member such that one end of the access member is located at the predetermined location and the other end of the access member is accessible to a user; inserting an end of a first segment of a plunger comprising a plurality of segments into the end of the access member that is farthest from the predetermined location in the subject; urging the first segment towards the end of the access member positioned at the predetermined location in the subject to urge the material in the access member towards the predetermined location; inserting an end of an additional segment of the plurality of segments into the end of the access member that is farthest from the predetermined location; and urging the additional segment towards the end of the access member positioned at the predetermined location to thereby urge the material in the access member towards the predetermined location, where the step of pushing additional segments through the access member may be repeated until the correct amount of material is delivered to the predetermined location in the subject.

The methods of the present invention may be employed to deliver a material to a predetermined location in a subject where the subject comprises an animal. In an embodiment, the subject may be a mammal. For example, the subject may be a human.

The predetermined location may, in certain embodiments, comprise a body part within a living body. In an embodiment, the predetermined location may comprise a bone. In one embodiment, the predetermined location may comprise a bone interior. For example, the predetermined location may comprise a portion of a spine. Thus, in one embodiment, the plunger may be sized for urging a material through an access member to thereby deliver the material to a bone interior, such as a vertebral body or disc of a spine.

For example, due to various traumatic or pathologic conditions, such as osteoporosis, a vertebral body can experience a vertebral compression fracture (VCF). Thus, the methods of the present invention may by used to repair a vertebral body lost due to a fracture, or when other degeneration occurs. The methods of the present invention are not limited in application to vertebrae, and may be used to repair other parts of a living or non-living organism. For example, in certain embodiments, methods of the present invention can be deployed in other bone types and within or adjacent other tissue types, such as in a vertebral disc, an arm bone, a leg bone, a knee joint, or the like.

The access member may provide a path to access a region or a body part that is located within a subject's body. For example, the access member may be designed to extend from an internal body part in a subject to outside of the subject's body. The access member may comprise an elongated hollow member such as a hollow tube. In an embodiment, the segments of the plunger and the access member are substantially cylindrical in shape. Or, the access member may be other shapes (e.g., polygonal) as described herein. For example, the access member may comprise a cannula, such as a cannula used to deliver a material to bone or another type of body part.

In an embodiment, the access member may be configured to provide percutaneous surgical access from outside of the subject to the predetermined location. In alternate embodiments, the percutaneous surgical access may comprises an incision ranging from about 0.1 to 4.0 centimeters (cm) in diameter, or from about 0.2 to 2.0 cm in diameter, or from about 0.25 to about 1 cm in diameter. Thus, in alternate embodiments, the percutaneous surgical access may comprise an incision that is less than 4 cm in diameter, or less than 2 cm in diameter, or less than 1 cm in diameter. In one example embodiment, the percutaneous surgical access may comprise an incision of about 1 cm in diameter. For example, in a typical percutaneous surgical repair of a spine, a cannula may establish a percutaneous path along its elongated axis to a vertebral body of one of the several vertebrae.

In an embodiment, each of the segments of the plunger used with the methods of the present invention comprises a diameter less than the internal diameter of the access member and a length less than the length of the access member. In this way, the plurality of segments may be inserted into the access member and function to push a material that has been loaded in the access member from one end of the access member to the other end of the access member. Also, in an embodiment, the plurality of the segments together comprise a length that is less than, or substantially the same as, the length of the access member. For example, a plunger comprising three segments may have an overall length that is less than the length of a tube comprising a material for delivery to a body part. In this way, the plunger will not extend out the distal end of the tube after the plunger has been used to urge the material in the tube out the distal end of the tube for delivery to a body part.

The plurality of segments used in the methods of the present invention may be connected as one unit, or may be separate parts. Thus, in an embodiment, at least two of the segments are attached to each other by a connector. In alternate embodiments, the connector may be fixedly or removably attached to the segments. The connector may, in certain embodiments, comprise a material that is bendable. This may allow the plunger to assume various conformations as the segments are urged through the tube. Also, in an embodiment, the connector may comprise a material having shape memory. By having shape memory, a connector having a first conformation may be urged into a second conformation, and then regain its original (first) conformation as the plunger is urged through the tube. Additionally or alternatively, it may be important for the connector to provide structural strength and to maintain an overall linear conformation as the plunger urges the material for delivery to the body part through the tube. Thus, in an embodiment, the connector may comprise a substantially rigid material. In an embodiment, the connector is substantially straight.

Alternatively, the plunger used in the methods of the present invention may comprise a plurality of segments that are not connected to each other. For example, in one embodiment, the plurality of segments may be configured such that an end of a first segment can be positioned to be in direct communication with an end of an adjacent segment.

In an embodiment, the segmented plunger used in the methods of the present invention may provide a means to deliver a material to a predetermined location in a subject where the working environment comprises limited access. Thus, embodiments of the methods of the present invention may further comprise at least one tool for urging at least one of the segments through the access member. The tool may be used to urge the segments through the access member either individually, or as a linear unit of segments. In an embodiment, the tool may be configured to be in communication with an end of at least one of the plurality of segments. In alternate embodiments, the tool may either be fixedly attached to the segment, or the tool may be removably attached to the segment.

The tool may comprise various shapes depending upon the specific application for which the method of the present invention is being used. In one example embodiment, the tool may comprise substantially flat surface configured to engage a surface of one of the segments. Also, the tool may be differently configured depending upon how it is used to urge segments through the access member. For example, in an embodiment, the tool may configured to engage the distal end of a segment that extends from the proximal end of the access member to push the segment towards the distal end of the access member. Alternatively, the tool may comprise a configuration designed to pull segments that are outside of an access member into the access member.

In an embodiment, the tool may comprise a handle. Alternatively or additionally, the tool may comprise a substantially planar conformation. In an alternate embodiments, the tool may comprise a substantially planar conformation that is rectangular, circular, or oval in shape. In an embodiment, the tool comprises a disk-like shape. Also, in an embodiment, the tool for urging segments through an access member may comprise an aperture or other means for the tool to be positioned around a connector connecting a first segment to a second segment. The aperture may allow the user to bend the plunger as it is being used to urge material through the access member. For example, the aperture may comprise a funnel-like shape having a wide opening at one end to allow for bending of the connector that extends from the access member, but having a straight portion at the other end to align the distal end of the connector with the access member. Also, the tool may be shaped to allow for a user to easily manipulate the tool. Thus, the tool may comprise a handle or a surface shaped to engage at least a portion of a user's hand.

For example, in an embodiment, the method may comprise the step of loading a material to be emplaced at a predetermined location in a subject in at least one access member. In an embodiment, the access member may comprise a cylindrical unit designed to have one end positioned at the predetermined location. In an embodiment, the access member may comprise a hollow tube. For example, the access member may comprise a cannula. At this point, the method may additionally comprise the step of positioning the access member so as to have one end near at the location of interest and the other end accessible to a user. In an embodiment, the access member may be threaded through a second cannula that has one end positioned at the predetermined location. Next, the distal end of a segmented plunger may be inserted into the proximal end of the access member. To deliver the material to the body part of interest, the proximal end of the segmented plunger may be pushed from the proximal end of the access member towards the distal end of the access member, such that the material to be delivered to the location of interest is urged through the access member. The methods of the present invention may be performed in conjunction with additional methods used in the art. For example, in one embodiment, the methods of the present invention may be used subsequent to accessing a bone and creating a cavity in a bone using a cannula-based drill and balloon catheter as described in U.S. Pat. No. 6,241,734.

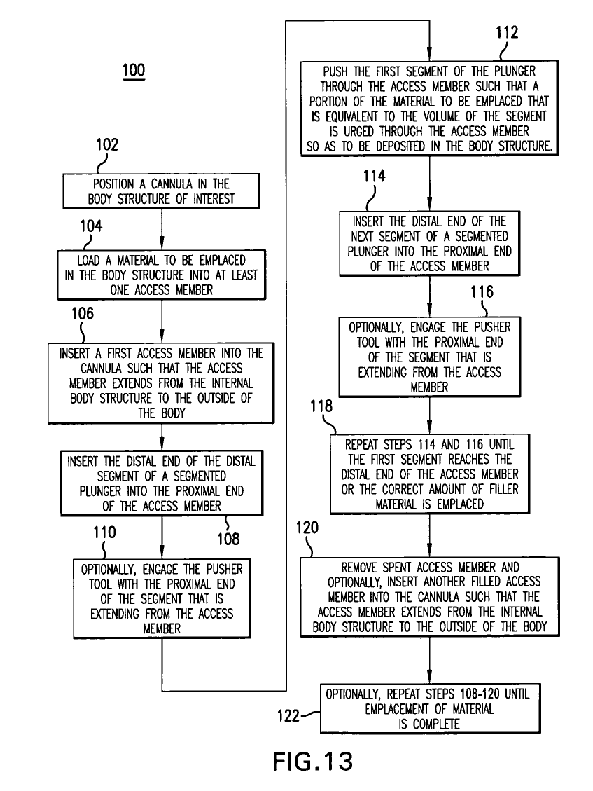

An embodiment of a method 100 of the present invention is shown in FIG. 13. For example, the method may comprise a first step 102 of positioning a cannula in the body part of interest. The canula, like the access member, may be configured to provide percutaneous surgical access to a body region or body part of interest. For example, for repair of a bone, the cannula may be used to position a drill bit to cut an opening in the bone to be repaired. Also, the cannula may be used to deliver a balloon catheter to the bone to further stabilize the bone by compacting any loose tissue at the site of repair.

Next, a material to be emplaced in the body part of interest may be loaded into at least one access member 104. For example, for delivery of a bone filler material to a vertebral bone, about 6-8 access members each comprising about 1.5 cc of bone filler may be used.

At this point, one of the access members comprising the material to be emplaced may be threaded through the outer cannula to the body part of interest 106. Next, the distal end of the distal (first) segment of the segmented plunger may be inserted in the proximal end of the access member 108. To expel the material from the distal end of the access member, the segmented plunger is pushed through the access member 112. In an embodiment, a pusher tool as described above is used to push the first segment through the access member 110. Or, a handle may be attached to the segment and used to push the segment through the access member. At this point, an additional segment may be inserted into the access member pushed through the access member to emplace additional material 114, optionally, using a pusher tool 116 or some other means. A plurality of segments may be pushed through the cannula until all of the material in the access member has been emplaced 118. As described above, a pushing tool may be used to urge each of the segments through the access member. Alternatively or additionally, a handle may be used to urge at least some of the segments through the access member.

Once all of the material in the first access member has been emplaced in the body part of interest, the spent (i.e., substantially empty) access member may be removed from the cannula and another access member positioned to have its distal end inserted in the body part of interest 120. The material in the second access member may then be delivered to the body part in a manner substantially as described for the first access member by repeating steps 108-120. Once the material in the second access member has been delivered to the internal body part of interest, additional access members may be used until the correct amount of material is delivered to the body part of interest 122.

In other embodiments, the present invention may comprise a method of providing a product, system, or kit for delivery of a material to a predetermined location in a subject. For example, in one embodiment, the method may comprise providing a product for delivery of a material to a predetermined location in a subject, where the method comprises manufacturing a plurality of segments sized for urging a material through an access member. In an embodiment, the method may comprise manufacturing a plunger comprising a plurality of segments sized for urging a material through an access member, where the access member comprises a path for delivering the material to a predetermined location within a subject.

In an embodiment, the segments and the access member are cylindrical in shape. Or, the access member and segments may be other shapes, such as oval, rectangular, polygonal (e.g., hexagonal, octagonal) and the like. For example, in an embodiment, the access member comprises a cannula. The plunger segments may be solid, or they may be at least partially hollow. In an embodiment, the individual segments are constructed so as to be sufficiently rigid such that they do not bend when inserted into the access member used to contain the material being delivered to the body location of interest. Also, the plunger segments may comprise a material that is compatible with the other parts of the system. Thus, the segments may be made of metal. For example, metals such as stainless steel, spring steel, nickel titanium alloys, other alloys, or aluminum may be used. Or, the segments may be made of plastic. For example, a resilient plastic such as vinyl, nylon, polypropylene, polyethylene, ionomer, polyurethane, or polyethylene tetraphthalate (PET) may be used. Again, one of ordinary skill in the art having the benefit of this disclosure would appreciate that other materials, including those that are well-known to one in the art, may be applied to configure the segments described herein.

As described above, the size of a plunger of the present invention may be such that each of the plunger segments comprises a diameter less than the internal diameter of the access member and a length less than the length of the access member. In this way, the plurality of segments may be inserted into the access member and function to push a material that has been loaded in the access member from one end of the access member to the other end of the access member. Also, in an embodiment, the plurality of the segments together comprise a length that is less than, or substantially the same as, the length of the access member. Thus, where the product comprises a plunger for a bone filler device, an access member (e.g., a cannula for accessing the bone) may comprise dimensions on the order of about 2 to 20 inches (50.8 mm to 508 mm), or about 4 to 15 inches (101 mm to 381 mm), or about 6 to 12 inches (152 mm to 305 mm), or about 8 inches (203 mm) in length. Also, in alternate embodiments, the cannula may range from about 0.05 to 0.5 inches (1.27 mm to 12.7 mm), or from about 0.1 to 0.3 inches (2.5 mm to 7.6 mm), or from about 0.12 to about 0.2 inches (3.1 mm to 5.1 mm), or from about 0.140 to 0.160 inches (3.6 mm to 4.1 mm) in diameter. Thus, the segmented plunger may comprise a total length that is about the same as the cannula, with a slightly smaller diameter, e.g., about 0.04 to 0.45 inches (1.0 mm to 11.4 mm), or from about 0.09 to about 0.29 inches (2.3 mm to 7.4 mm), or from about 0.1 to about 0.19 inches (2.5 mm to 4.8 mm), or from about 0.13 to 0.15 inches (3.3 mm to 3.8 mm). Also, in alternate embodiments, and depending upon the length and width of the plunger, the number of segments per plunger may vary from about 2 to 20, or from about 2 to 10, or from 2 to 5, or 2 to 4, or 2 to 3. For example, the plunger may be sized to fit pre-existing bone filler devices such as the KYPHX®EXPRESS™ bone filler device that is commercially available from Kyphon, Inc.

The method may additionally comprise linking the individual segments to form a plurality of connected segments. For example, where the segments comprise metal, the connector may be soldered to the ends of each segment. Or, where the segments comprise a plastic, the connectors may be molded onto, or made as a part of, the segments. Or, the connectors may comprise a fitting such that a connector from one segment may be inserted into a holder or fastener on the end of a second segment.

As described herein, the connector segment(s) may comprise a material that is flexible, such that the connector itself is able to bend, or the connector may comprise a material that is substantially rigid. Also, in an embodiment, the connector may comprise a material that comprises shape memory, such as nitinol. Thus, in alternate embodiments, the connectors may be made of aluminum, stainless steel, spring steel, nickel titanium alloys, or other alloys. Or, in some embodiments, the connectors may be made of plastic. For example, a resilient plastic such as vinyl, nylon, polypropylene, a polyethylene, ionomer, polyurethane, and polyethylene tetraphthalate (PET) may be used. In alternate embodiments, and depending upon the length of the plunger, the connectors may range in length from about 0.2 cm to 5 cm, or from about 0.5 cm to 2 cm, or from about 0.8 cm to about 1 cm in length. Also, in alternate embodiments, and depending upon the length and width of the plunger, the connectors may range in diameter from about 0.1 mm to 5 mm, or from about 0.2 mm to 3 mm, or from about 1 to 2 mm. Also, depending upon the number of segments, the number of connectors per plunger may vary from about 1 to 19, or from about 1 to 9, or from 1 to 4, or 1 to 3, or 1 to 2.

The method may further comprise making a tool for engaging an individual segment and pushing it through an access member. In one embodiment, the tool may comprise a substantially planar shape, such as a disk. The tool may shaped to comprise a recessed portion that can engage the end of one of the cylindrical segments. Also, the tool may be shaped to comprise a slit, or other type of aperture, so the disk can be wrapped around one of the connectors. As described herein, the tool may also be shaped to comprise an opening allowing for bending of the connector that extends outside of an access member. The tool for urging segments through an access member may be made of a material that is compatible with the other parts of the system. For example, the tool may be cast from a metal such as aluminum, stainless steel, spring steel, nickel titanium alloys, or other metal alloys. Or, the tool may be made of medical grade plastic, such as vinyl, ionomer, polypropylene, polyethylene, PET, or nylon. The tool may be shaped to fit between connected segments, or where there is limited clearance. For example, in alternate embodiments, a disk-shaped tool may comprise a diameter in the range of from about 2 mm to 80 mm, or from about 10 mm to about 40 mm, or about 25 mm. Also, in alternate embodiments, a disk-shaped tool may comprise a height (e.g., size along the length of the access member and/or connectors) in the range of from about 2 mm to 20 mm, or from about 3 mm to about 10 mm, or about 4 mm.

The method of making the device and/or system of the present invention may also comprise the step of making a handle to help push the segments through an access member. The handle may be configured to attach to the proximal end of at least one of the segments to allow an individual to urge the segment through the access member by pushing on the handle. The handle may permanently attach to the segment, or it may removably attach to the segment. Similar to the pushing tool, the handle may be made of a material that is compatible with the other parts of the system. For example, the handle may be made of metal such as aluminum, stainless steel, spring steel, nickel titanium, or other metal alloys. Or, the handle may be made of a hard plastic, such as polyurethane, polystyrene polycarbonate, or nylon.

The method may further comprise the step of manufacturing an access member to be used with the plurality of segments. As described herein, the access member may provide a path to access a region or a body part that is located within a subject's body. The access member may be any type of device that can extend from the location of interest (e.g., a bone or an organ) to be accessible to an individual accessing the location of interest. For example, the access member may be designed to extend from an internal body part in a subject to outside of the subject's body. The access member may comprise an elongated hollow member such as a hollow cylinder or a tube. For example, the access member may comprise a cannula, such as a cannula used to deliver a material to bone or another type of body part. The access member may be constructed, for example, using standard, flexible, medical grade plastic materials, such as vinyl, ionomer, polypropylene, polyethylene, PET, or nylon. In some embodiments, the access member may comprise a metal. Thus, in alternate embodiments, the access member, like other parts of the system, may comprise aluminum, stainless steel, spring steel, nickel titanium, or other metal alloys. Sizes for the access member may depend on the body location being access. Thus, in alternate embodiments, where the product comprises a plunger for a bone filler device, an access member may comprise dimensions on the order of about 2 to 20 inches (50.8 mm to 508 mm), or about 4-15 inches (101 mm to 381 mm), or about 6 to 12 inches (152 mm to 305 mm), or about 8 inches (203 mm) in length. Also, in alternate embodiments, the access member may range from about 0.05 to 0.5 inches (1.27 mm to 12.7 mm), or from about 0.140-0.160 inches (3.6 mm to 4.1 mm) in diameter. For example, the plunger may be sized to fit pre-existing bone filler devices such as the KYPHX®EXPRESS™ bone filler device that is commercially available from Kyphon, Inc.

Each of the components used in the products, systems, and kits of the present invention may comprise a material that may be sterilized by either chemical treatment, high temperature, and/or high pressure, exposure to sterilizing gas, or a combination of sterilization treatments as is known in the art. Also, the components of the products, systems, and kits of the present invention may be disposable, or may be formulated to allow for cleaning, re-sterilization, and re-use.

Embodiments of the present invention may provide certain advantages. For example, in an embodiment, using the segmented plunger of the present invention may provide access to a body part where the clearance is limited. As described above, emplacement of a material at a location of interest in a subject, such as emplacing a bone filler material in bone, may require monitoring by fluorography, which in turn can require positioning the arm of an X-ray machine close to the subject's torso. Using a segmented plunger of the present invention in a bone filler device may allow for a physician to emplace bone filler material where clearance to insert a straight rod-like plunger is restricted due to the positioning of X-ray equipment, or for other reasons.

Also, in certain embodiments, the present invention may allow for increased flexibility in the amount of material that is delivered to a body location of interest. For example, because the segmented plunger of the present invention is substantially modular in nature, plungers of varying length may easily be assembled and used for delivery of a material to the location of interest.

It will be understood that each of the elements described above, or two or more together, may also find utility in applications differing from the types described. While the invention has been illustrated and described as devices, systems, kits and methods to deliver a material to an internal body part, it is not intended to be limited to the details shown, since various modifications and substitutions can be made without departing in any way from the spirit of the present invention. Where method and steps describe above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. As such, further modifications and equivalents of the invention herein disclosed may occur to persons skilled in the art using no more than routine experimentation, and all such modifications and equivalents are believed to be within the spirit and scope of the invention as described herein. All patents and published patent applications referred to in this document are incorporated by reference in their entireties as if each individual publication or patent application were specifically and individually put forth herein.

That which is claimed is:

1. A product comprising a plunger having a plurality of segments sized for urging a material through an access member to a predetermined location within a subject.

2. The product of claim 1, wherein the predetermined location comprises a bone interior.

3. The product of claim 1, wherein the predetermined location comprises at least one of a vertebral body or spinal disc.

4. The product of claim 1, wherein the access member is configured to provide percutaneous surgical access to the predetermined location.

5. The product of claim 1, wherein each of the segments comprises a diameter less than the internal diameter of the access member and a length less than the length of the access member.

6. The product of claim 5, wherein the plurality of the segments together comprise a length less than, or substantially the same as, the length of the access member.

7. The product of claim 1, wherein the access member comprises a tube.

8. The product of claim 1, wherein at least two of the segments are attached to each other by a connector.

9. The product of claim 8, wherein the connector comprises a bendable material.

10. The product of claim 8, wherein the connector comprises a material having shape memory.

11. The product of claim 8, wherein the connector comprises a substantially rigid material.

12. The product of claim 1, further comprising at least one tool for urging at least one of the segments through the access member.

13. The product of claim 12, wherein the tool is configured to be in communication with an end of at least one of the plurality of segments.

14. The product of claim 12, wherein the tool is configured to have an aperture, the aperture allowing the tool to be positioned around a connector connecting a first segment to a second segment.

15. A system for delivering a material to a predetermined location in a subject comprising:
(a) an access member; and
(b) a plunger having a plurality of segments each having a diameter less than the internal diameter of the access member and a length less than the length of the access member.

16. The system of claim 15, wherein the access member is configured to provide percutaneous surgical access to the predetermined location.

17. The system of claim 15, wherein the predetermined location comprises a bone interior.

18. The system of claim 15, wherein the predetermined location comprises at least one of a vertebral body or a spinal disc.

19. The system of claim 15, wherein the plurality of the segments together comprise a length less than, or substantially the same as, the length of the access member.

20. The system of claim 15, further comprising a material to be delivered to the predetermined location emplaced within at least a portion of the access member.

21. The system of claim 15, wherein at least two of the segments are attached to each other by a connector.

22. The system of claim 15, further comprising at least one tool configured to be in communication with an end of at least one of the plurality of cylindrical segments for urging at least one of the segments of the plurality of segments through the access member.

23. The system of claim 22, wherein the tool is configured to have an aperture, the aperture allowing for the tool to be positioned around a connector connecting a first segment to a second segment.

24. The system of claim 15, wherein the access member comprises a tube having a distal opening that is not linearly aligned with the proximal opening.

25. A kit for delivering a material to a predetermined location in a subject comprising:
(a) an access member;
(b) a plunger having a plurality of segments each having a diameter less than the internal diameter of the access member and a length less than the length of the access member; and
(c) instructions for use.

26. The kit of claim 25, wherein the plurality of the segments together comprise a length that is less than, or substantially the same as, the length of the access member.

27. The kit of claim 25, wherein the predetermined location comprises a bone interior.

28. The kit of claim 25, wherein the predetermined location comprises at least one of a vertebral body or a spinal disc.

29. The kit of claim 25, wherein the access member is configured to provide percutaneous surgical access to the predetermined location.

30. The kit of claim 25, wherein at least two of the segments are attached to each other by a connector.

31. The kit of claim 25, further comprising a container for holding each of the parts under sterile conditions, or for transporting the parts from a first site to a second site.

32. A method for delivery of a material to a predetermined location in a subject comprising the steps of:
inserting an end of a first segment of a plunger comprising a plurality of segments into the end of an access member, the access member comprising at least a portion of the material to be delivered to the predetermined location, and the segments each comprising a diameter less than the internal diameter of the access member; and
urging the segment at least partially through the access member.

33. The method of claim 32, further comprising inserting an end of an additional segment of the plurality of segments into the end of the access member, and urging the additional segment through the access member.

34. The method of claim 32, wherein the predetermined location comprises a bone interior.

35. The method of claim 32, wherein the predetermined location comprises at least one of a vertebral body or a spinal disc.

36. The method of claim 32, wherein the access member is configured to provide percutaneous surgical access to the predetermined location.

37. The method of claim 32, wherein the plurality of the segments together comprise a length that is less than, or substantially the same as, the length of the access member.

38. A method of providing a product for delivery of a material to a predetermined location in a subject comprising manufacturing a plurality of segments sized for urging a material through an access member.

39. The method of claim 38, wherein the predetermined location comprises a bone interior.

40. The method of claim 38, wherein the predetermined location comprises at least one of a vertebral body or a spinal disc.

41. The method of claim 38, wherein the access member is configured to provide percutaneous surgical access to the predetermined location.
